(19) United States
(12) Patent Application Publication
(10) Pub. No.: US 2008/0243161 A1
(43) Pub. Date: Oct. 2, 2008

(54) Disposable lancet with re-cocking prevention means

(76) Inventor: Ramzi Abulhaj, Miramar, FL (US)

Correspondence Address: Frank L. Kubler, 13261 S.W. 54th Court, Miramar, FL, 33027, US

(21) Appl. No.: 11/731,470

(22) Filed: Mar. 30, 2007

(51) Int. Cl. A61B 17/14

(52) U.S. Cl. 606/182; 606/181

(57) ABSTRACT

A lancet includes a proximal mounting platform; a shuttle biasing spring extending distally from the mounting platform; a shuttle having distally protruding needle, the shuttle being mounted on the shuttle biasing spring; a housing first section having a tubular first section side wall engagingly fitted to the mounting platform and fitted in telescopic relation to a tubular second section side wall of a housing second section which has a distal needle passing opening; a shuttle cocking mechanism including a shuttle shoulder having a shoulder distal face, two spaced apart and resilient shuttle engaging arms extending distally from the mounting platform and tilted toward each other and having inwardly protruding grip fingers positioned to fit over the shoulder distal face and thus engage the shuttle shoulder, and having outwardly ramped distal arm tips obstructing and deflecting re-entry of the shuttle between the engaging arms.

### BACKGROUND OF THE INVENTION

1. Field of the Invention

The present invention relates generally to the field of lancet devices. More specifically the present invention relates to a disposable lancet with a mechanism for preventing re-cocking. The lancet includes a proximal mounting platform, a shuttle having a distally protruding needle and being mounted on a shuttle biasing spring abutting and extending distally from the mounting platform, a housing first section having a tubular first section side wall engagingly fitted to the mounting platform, a housing second section having a distal needle passing opening and having a tubular second section side wall fitted in telescopic relation to the first section side wall, a shuttle cocking mechanism for releasibly holding the shuttle against the biasing spring while the spring is compressed, and a re-cocking prevention mechanism for preventing the shuttle from being pushed against the spring to compress the spring following release of the shuttle from the shuttle re-cocking mechanism.

The shuttle cocking mechanism preferably includes a shuttle shoulder, two spaced apart and resilient shuttle engaging arms extending distally from the mounting platform and tilted toward each other and having inwardly protruding grip fingers positioned to fit over the shuttle shoulder distal face of and thus engage the shuttle shoulder, and a proximally and centrally converging taper portion of the second section side wall positioned to abut and wedgingly fit between and separate the shuttle engaging arms as the housing first and second sections are telescopingly collapsed together. The re-cocking prevention mechanism includes inward resilient biasing of the engaging arms, combined with outward ramping of the distal arm tips so that the distal arm tips obstruct and deflect re-insertion of the shuttle between the engaging arms, thus preventing re-cocking.

The tubular second section side wall preferably slidably and telescopingly fits within the tubular first section side wall. The housing second section preferably includes the second section tubular side wall and an end wall provided by a separable second section distal housing end structure removably and engagingly fitted into the second section tubular side wall distal end, the distal housing end structure having the needle passing opening.

2. Description of the Prior Art

There have long been disposable lancets having a lancet housing and a needle mounted to a spring-loaded needle shuttle within the housing, for propelling a needle tip into the skin of a user and then automatically retracting the needle back into the lancet housing. A problem with most of these prior disposable lancets has been that they can be re-cocked and reused, which is an unsanitary and potentially hazardous practice. One prior disposable lancet having re-cocking prevention means is that of Levin, et al., U.S. Pat. No. Re. 32,922 issued on May 16, 1989 for a blood sampling instrument. Levin, et al., includes a tubular member containing the working parts and from which flange extends radially outward so that the instrument can be held easily by a patient and pressed against a finger while releasing the trigger mechanism.

Macors, et al., U.S. Pat. No. 5,282,822, issued on Feb. 1, 1994 discloses a lancet ejector for a lancet injector. Macors, et al., includes a generally cylindrical portion or slide having a lancet holding member connected to the distal end of the slide and a cylindrical extension or spring connector at the proximal end of the slide, and a biasing spring.

Kim, U.S. Pat. No. 5,707,384, issued on Jan. 13, 1998 teaches a lancet device for obtaining blood samples. Schraga, U.S. Pat. No. 6,258,112, issued on Jul. 10, 2001 and Schraga, U.S. Pat. No. 6,514,270, issued on Feb. 4, 2003 both reveal a single use lancet device. Schralga, U.S. Pat. No. 6,949,111, issued on Sep. 27, 2005 further teaches a lancet device. And finally Crossman, U.S. Pat. No. 6,719,771 issued on Apr. 13, 2004 discloses a blood sampling device.

It is thus an object of the present invention to provide a disposable lancet with a re-cocking prevention mechanism which is very simple in construction and easy to manufacture, and yet is entirely reliable.

### SUMMARY OF THE INVENTION

The present invention accomplishes the above-stated objectives, as well as others, as may be determined by a fair reading and interpretation of the entire specification.

A lancet is provided, including a proximal mounting platform; a shuttle biasing spring extending distally from the mounting platform; a shuttle having distally protruding needle with a pointed needle distal end, the shuttle being mounted on the shuttle biasing spring; a housing first section having a tubular first section side wall engagingly fitted to the mounting platform; a housing second section having a distal needle passing opening and having a tubular second section side wall fitted in telescopic relation to the first section side wall; a shuttle cocking mechanism including a shuttle shoulder having a shoulder distal face, two spaced apart and resilient shuttle engaging arms extending distally from the mounting platform and tilted toward each other and having inwardly protruding grip fingers positioned to fit over the shoulder distal face and thus engage the shuttle shoulder; a proximally, converging taper portion of the second section side wall positioned to abut and wedgingly fit between and separate the shuttle engaging arms as the housing first and second sections are telescopingly collapsed together, and a re-cocking prevention mechanism including the inward resilient biasing of the engaging arms combined with outwardly ramped distal arm tips obstructing and deflecting re-entry of the shuttle between the engaging arms. ABSOLUTELY NEED RAMPED TIPS AS WELL ???

The tubular second section side wall preferably slidably and telescopingly fits within the tubular first section side wall. The housing second section preferably includes the second section tubular side wall and a separable second section distal housing end structure removably and engagingly fitted into the second section tubular side wall distal end, the distal housing end structure having the needle passing opening. The distal housing end structure preferably includes a side wall with a helical cam segment slot and the tubular second section side wall includes an inwardly protruding follower pin sized and positioned to fit into the helical segment slot, so that rotating the distal housing end structure relative to the second section side wall causes the inwardly protruding follower pin to enter and engage the helical cam segment slot. The lancet preferably additionally includes a closure lid fitting snugly and removably over the housing second section distal end for safe storage and transport of the lancet.

The shuttle preferably includes a circular shuttle cylinder into which the needle is axially embedded so that the pointed needle distal end protrudes distally from the shuttle cylinder, and where the shuttle shoulder includes a shoulder flange protruding radially from the shuttle cylinder. The shuttle biasing spring preferably is a coil spring having a spring proximal end embedded in the mounting platform and having a spring distal end fitted around and thus encompassing a portion of and attached to the shuttle.

### BRIEF DESCRIPTION OF THE DRAWINGS

Various other objects, advantages, and features of the invention will become apparent to those skilled in the art from the following discussion taken in conjunction with the following drawings, in which:

FIG. 1 is a cross-sectional side view of the preferred embodiment of the present disposable lancet, including the closure lid.

FIG. 2 is a side view of the disposable lancet of FIG. 1.

FIG. 3 is an exploded view of the disposable lancet of FIG. 1 from a distal perspective.

FIG. 4 is an exploded view of the disposable lancet of FIG. 3 from a proximal perspective.

FIG. 5 is a side view of the proximal mounting platform and shuttle engaging arms.

FIG. 6 is a side view of the preferred shuttle, showing the embedded needle in broken lines.

FIG. 7 is a lateral projection of the side view of FIG. 6, again with the embedded needle in broken lines.

FIG. 8 is a cross-sectional side view of housing first section.

FIG. 9 is a side view of the distal housing end structure.

FIG. 10 is a side view of the closure lid.

FIG. 11 is a lateral projection of the side view of FIG. 10.

FIG. 12 is cross-sectional side view of the closure lid.

FIG. 13 is a side view of the lancet of FIG. 1.

FIG. 14 a side view of the lancet of FIG. 13, with the closure lid moved to and snapped onto the proximal end of the lancet housing.

FIG. 15 is a distal perspective view of the lancet of FIG. 13, with the contact pad forward-most, and ready for use.

FIG. 16 is a proximal perspective view of the lancet of FIG. 15.

FIG. 17 is a perspective view of the lancet of FIG. 16 being gripped by user hands and the closure lid removed.

FIG. 18 is a perspective view of the lancet of FIG. 17 with the contact pad pressed against a finger of a user hand and the other user hand pressing the lancet toward the user finger to trigger the release of the shuttle and needle to pierce the skin of the user finger.

FIG. 19 is a view as in FIG. 18, after release of the shuttle and needle.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

### First Preferred Embodiment

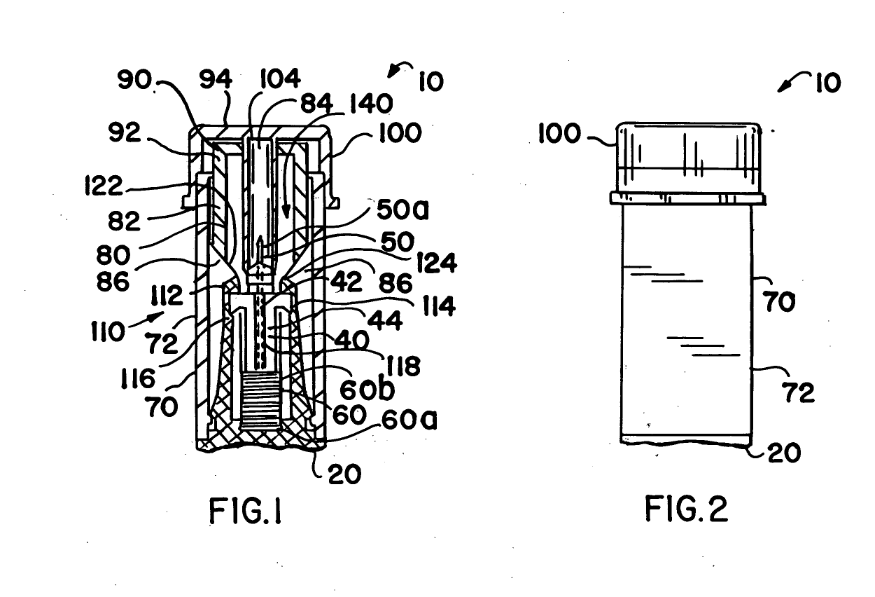

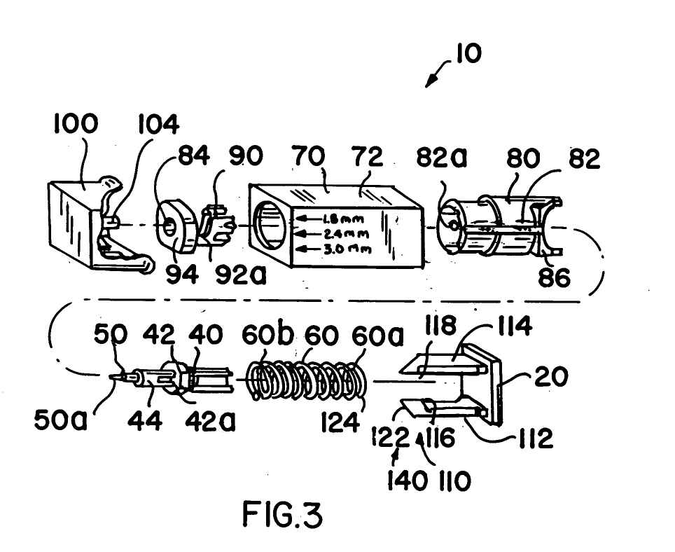

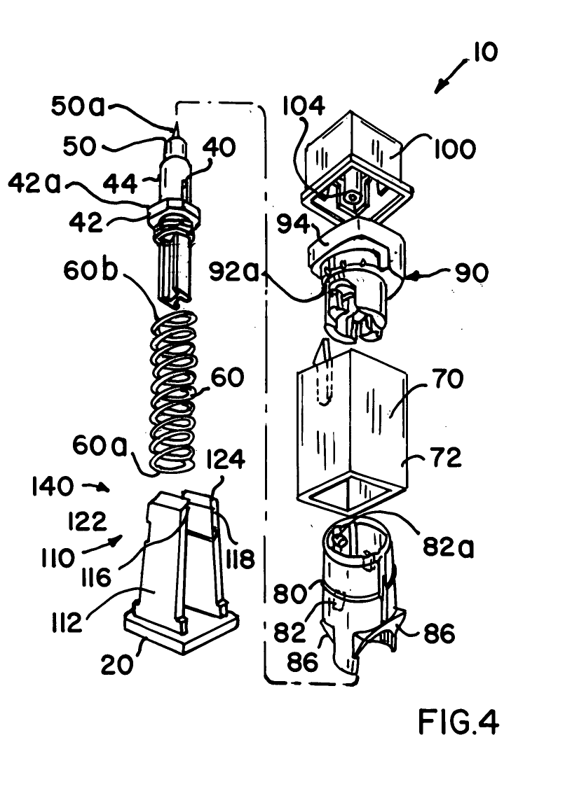

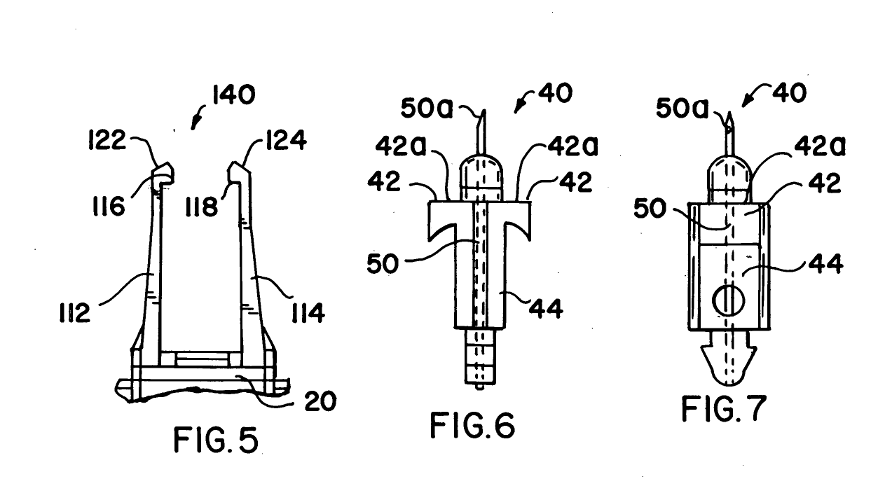

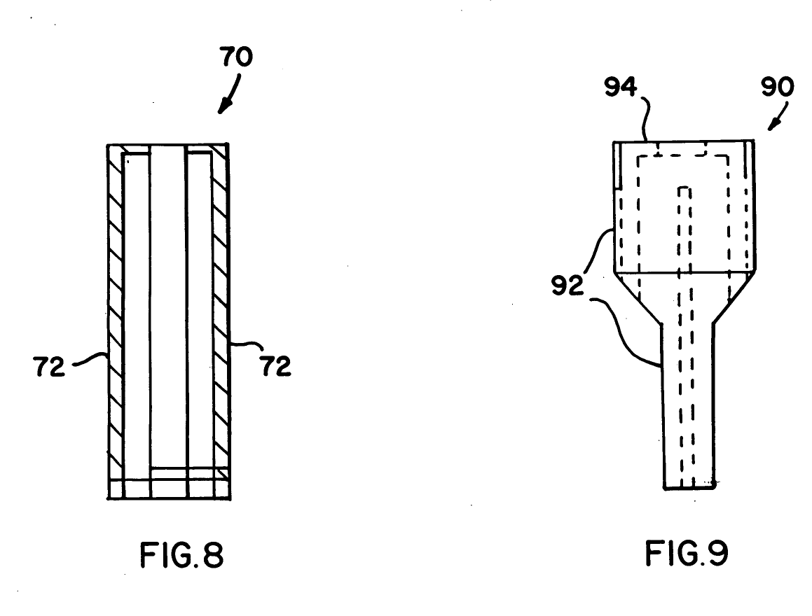

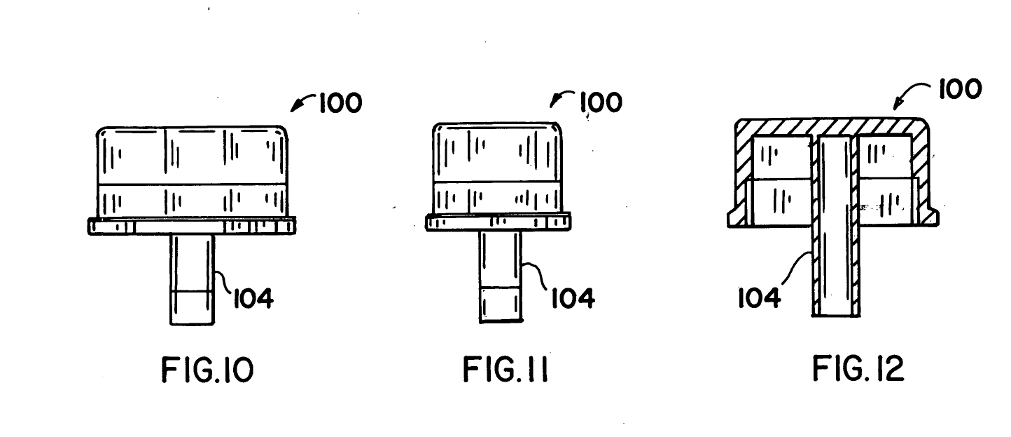

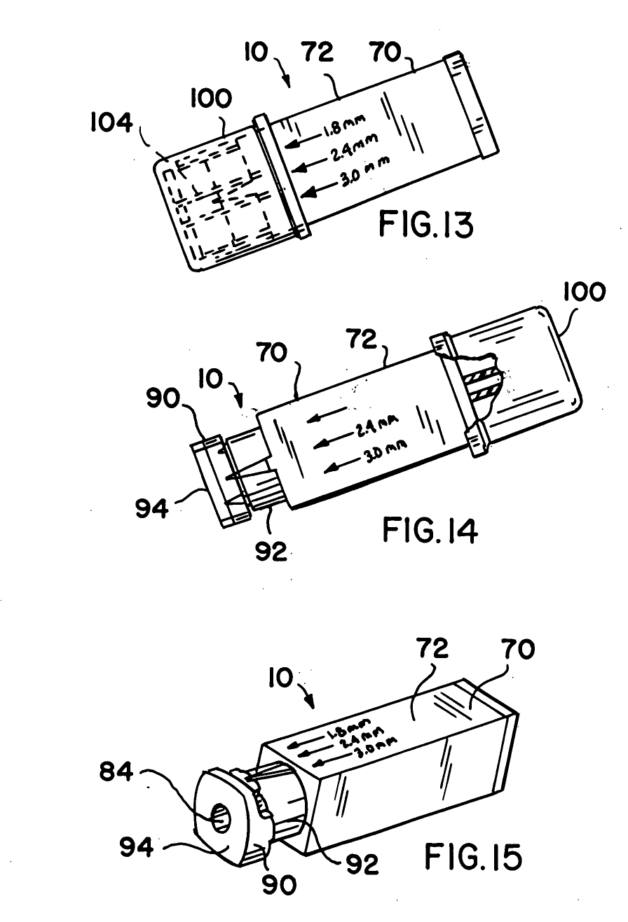

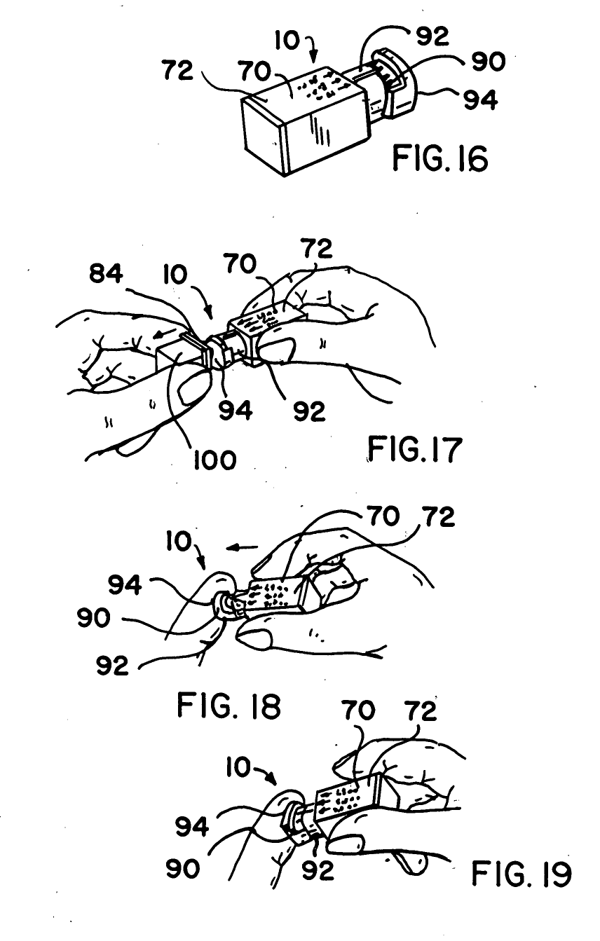

Referring to FIGS. 1-19, a disposable lancet 10 is disclosed including a proximal mounting platform 20, a shuttle 40 having a distally protruding needle 50 and being mounted on a shuttle biasing spring 60 abutting and extending distally from the mounting platform 20, a housing first section 70 having a tubular first section side wall 72 engagingly fitted to the mounting platform 20, a housing second section 80 having a distal needle passing opening 84 and having a tubular second section side wall 82 fitted in telescopic relation with the first section side wall 72, a shuttle cocking mechanism 110 for releasibly holding the shuttle 40 against the biasing spring 60 while the spring 60 is compressed, and a re-cocking prevention mechanism 140 for preventing the shuttle 40 from being pushed against the spring 60 to compress the spring 60 again. The structure of the present invention is similar to that of the prior invention of applicant set forth in U.S. Pat. No. 6,852,119 issued on Feb. 8, 2005, except for the structure of the re-cocking prevention mechanism 140.

The shuttle cocking mechanism 110 preferably includes a shuttle shoulder 42 with a shoulder distal face 42a, two spaced apart and resilient shuttle engaging arms 112 and 114, respectively, extending distally from the mounting platform 20 and tilted toward each other and having inwardly protruding grip fingers 116 and 118 positioned to fit over the shoulder distal face 42a of and thus engage the shuttle shoulder 42, and a proximally and centrally converging taper portion 86 of the second section side wall 82 positioned to abut and wedgingly fit between and separate the shuttle engaging arms 112 and 114 as the housing first and second sections 70 and 80 are telescopingly collapsed together. The re-cocking prevention mechanism 140 includes the inward resilient biasing of the engaging arms 112 and 114, combined with outwardly ramped distal arm tips 122 and 124 so that the distal arm tips 122 and 124 obstruct and deflect re-insertion of the shuttle 40 between the engaging arms 112 and 114.

The tubular second section side wall 82 preferably slidably and telescopingly fits within the tubular first section side wall 72. The housing second section 80 preferably includes an end wall provided as part of a separable second section distal housing end structure 90 removably and engagingly fitted into the second section tubular side wall 82 distal end, the distal housing end structure 90 having the needle passing opening 84. The distal housing end structure 90 preferably has a housing distal contact pad 94 and has a cap side wall 92 with cap fastening means in the form of a helical cam segment slot 92a and the tubular second section side wall 82 preferably has an inwardly protruding follower pin 82a sized and positioned to fit into the helical cam segment slot 92a so that rotating the distal housing end structure 90 relative to the second section side wall 82 causes the inwardly protruding follower pin 82a to enter, slide helically within and engage the helical segment slot 92a in the manner of a screw thread. A closure lid 100 preferably fits snugly and removably over the housing second section 80 distal end for safe storage and transport of the lancet 10, the closure lid 100 having a needle passing opening engaging lid tubular stem 104 which fits snugly into the distal needle passing opening 84 when the closure lid 100 is fitted onto the housing first section side wall 72. The shuttle 40 preferably includes a circular shuttle cylinder 44 into which the needle 50 is axially embedded so that the pointed needle end 50a distally protrudes from the shuttle cylinder 44, and the shuttle shoulder 42 preferably takes the form of a shoulder flange protruding radially from the shuttle cylinder 44 including the shoulder flange distal face 42a. All parts of lancet 10, except for biasing spring 60 and needle 50, preferably are formed of a suitable plastic. The shuttle biasing spring 60 preferably is a coil spring having a spring proximal end 60a embedded in the mounting platform 20 and having a spring distal end 60b fitted around and thus encompassing a portion of and attached to the shuttle 40. The shuttle engaging arms 112 and 114 preferably are a monolithic part of and integral with the mounting platform 20.

### Method of Use and Operation

In practicing the invention, the following method may be used. The needle passing opening 84 of the distal end of the housing second section 80 is placed against the user skin, the housing first section 70 is pushed toward the user skin and toward the housing second section 80 so that the housing first section 70 and housing second section 80 telescopically collapse together, causing the proximally converging taper portion 86 of the second section side wall 82 to abut and wedgingly fit between and separate the shuttle engaging arms 112 and 114, to move the distal arm tips 122 and 124 off shuttle shoulder 42 and thus release the shuttle 40 so that the shuttle biasing spring 60 expands and drives the shuttle 40 distally beyond the distal arm tips 122 and 124, permitting engaging arms 112 and 114 to resiliently tilt inwardly, positioning the outwardly ramped distal arm tips 122 and 124 to obstruct and prevent re-insertion of the shuttle 40 between engaging arms 112 and 114.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

I claim as my invention:

1. A lancet, comprising:
a proximal mounting platform;
a shuttle biasing means connected to said proximal mounting platform;
a shuttle having a distally protruding needle with a pointed needle distal end, said shuttle being mounted on said shuttle biasing means;
a housing first section having a tubular first section side wall engagingly fitted to said mounting platform;
a housing second section having a distal needle passing opening and having a tubular second section side wall fitted in telescopic relation to said first section side wall;
a shuttle cocking mechanism including a shuttle shoulder having a shoulder distal face, two spaced apart and resilient shuttle engaging arms extending distally from said mounting platform and tilted toward each other and having inwardly protruding grip fingers positioned to fit over said shoulder distal face and thus engage said shuttle shoulder;
a proximally converging taper portion of said second section side wall positioned to abut and wedgingly fit between and separate said shuttle engaging arms as said housing first and second sections are telescopingly collapsed together,
and a re-cocking prevention means comprising outwardly ramped distal arm tips obstructing and deflecting re-entry of said shuttle between said engaging arms.

2. The lancet of claim 1, wherein said tubular second section side wall slidably and telescopingly fits within said tubular first section side wall.

3. The lancet of claim 1, wherein said housing second section comprises said second section tubular side wall and a separable second section distal housing end structure removably and engagingly fitted into said second section tubular side wall distal end, said distal housing end structure having said needle passing opening.

4. The lancet of claim 3, wherein said distal housing end structure comprises a side wall with a helical cam segment slot and said tubular second section side wall comprises an inwardly protruding follower pin sized and positioned to fit into said helical segment slot, such that rotating said distal housing end structure relative to said second section side wall causes the inwardly protruding follower pin to enter and engage the helical cam segment slot.

5. The lancet of claim 1, additionally comprising a closure lid fitting snugly and removably over said housing second section distal end for safe storage and transport of said lancet.

6. The lancet of claim 1, wherein said shuttle comprises a circular shuttle cylinder into which said needle is axially embedded such that said pointed needle distal end protrudes distally from said shuttle cylinder, and wherein said shuttle shoulder comprises a shoulder flange protruding radially from said shuttle cylinder.

7. The lancet of claim 1, wherein said shuttle biasing means is a coil spring having a spring proximal end embedded in said mounting platform and having a spring distal end fitted around and thus encompassing a portion of and attached to said shuttle.

8. The lancet of claim 1, wherein said shuttle engaging arms are a monolithic part of and integral with said mounting platform.

9. A lancet, comprising:
a proximal mounting platform;
a shuttle biasing means connected to said proximal mounting platform;
a shuttle having a distally protruding needle with a pointed needle distal end, said shuttle being mounted on said shuttle biasing means;
a housing first section having a tubular first section side wall engagingly fitted to said mounting platform;
a housing second section having a distal needle passing opening and having a tubular second section side wall fitted in telescopic relation to said first section side wall;
a shuttle cocking mechanism including a shuttle shoulder having a shoulder distal face, two spaced apart and resilient shuttle engaging arms extending distally from said mounting platform and tilted toward each other and having inwardly protruding grip fingers positioned to fit over said shoulder distal face and thus engage said shuttle shoulder;
a proximally converging taper portion of said second section side wall positioned to abut and wedgingly fit between and separate said shuttle engaging arms as said housing first and second sections are telescopingly collapsed together,
and a re-cocking prevention means comprising the inward resilient biasing of said engaging arms.
